(19) United States
(12) Patent Application Publication
(10) Pub. No.: US 2007/0244406 A1
(43) Pub. Date: Oct. 18, 2007

(54) Method and system for managing interventional pulmonology

(76) Inventors: Igal Kushnir, Pardes Hana (IL); Meir Botbol, Pardes Hana (IL); Alon Kushnir, Herev Le' et (IL)

Correspondence Address: NATH & ASSOCIATES, PLLC, 112 South West Street, Alexandria, VA, 22314, US

(21) Appl. No.: 11/581,745

(22) Filed: Oct. 17, 2006

Related U.S. Application Data
(60) Provisional application No. 60/728,334, filed on Oct. 20, 2005.

(51) Int. Cl. A61B 5/087

(52) U.S. Cl. 600/538

(57) ABSTRACT

The invention provides a method and system for assessing an interventional pulmonology procedure. A plurality of sound transducers are fixed on a surface of the individual over an individual's respiratory tract that generate signals indicative of pressure waves at the transducers. A processor receives the signals and generates from the signals an image indicative of airflow in at least a portion of the respiratory tract before the interventional pulmonology procedure is carried out. A second image indicative of airflow in at least a portion of the respiratory tract is then generated from the signals after the interventional procedure has been carried out. display the first and second sequences of images simultaneously on a display device. The first and second images are then displayed on a display.

This application claims the benefit of prior U.S. provisional patent application No. 60/728,334 filed Oct. 20, 2005, the contents of which are hereby incorporated by reference in their entirety.

### FIELD OF THE INVENTION

The invention relates to medical devices and methods, and more particularly to such devices and methods for carrying out an interventional procedure.

### BACKGROUND OF THE INVENTION

Interventional pulmonology is a field within pulmonary medicine focused on the use of bronchoscopic, pleuroscopic and other techniques for the treatment of thoracic disorders such as tracheobronchial stenosis and pleural effusions associated with malignant tumors. A number of techniques, such as rigid bronchoscopic recanalization, balloon dilation, laser bronchoscopy, cryotherapy, electrocautery, brachytherapy, photodynamic therapy, valve placement, and stent placement are in therapeutic use for the management of airway stenosis.

A careful pretreatment evaluation is necessary to identify the source of the airway obstruction and to select the course of treatment. Pulmonary-function testing (PFT) and thoracic imaging techniques such as computed tomorgraphy (CT) have been used in the evaluation of a patient with suspected obstruction of the central airways, though bronchoscopy is considered to be the diagnostic gold standard. Follow-up includes assessment of treatment success (i.e. recanalization/resection rate, rate of repeated interventions), complications (i.e. stent migration, perforation of the airway, fistula formation), assessment of clinical symptoms (i.e. relief of dyspnea), and clinical outcome such as pulmonary function test results.

Body sounds are routinely used by physicians in the diagnosis of various disorders. A physician may place a stethoscope on a person's chest or back and monitor the patient's breathing in order to detect adventitious (i.e. abnormal or unexpected) lung sounds. The identification and classification of adventitious lung sounds often provides important information about pulmonary abnormalities.

U.S. Pat. No. 6,887,208 to Kushnir et al. provides a system and method for recording and analyzing sounds produced by the respiratory tract. Respiratory tract sounds are recorded at a plurality of locations over an individual's thorax and the recorded sounds are processed to produce an image of the respiratory tract. The processing involves determining from the recorded signals an average acoustic energy, at a plurality of locations over the thorax over a time interval from t₁ to t₂. The term “acoustic energy” at a location is used herein to refer to a parameter indicative of, or approximating, the product of the pressure and the mass propagation velocity at that location. The image may be used to analyze respiratory tract physiology and to detect pathological conditions. Additionally, a time interval can be divided into a plurality of sub-intervals, and an average acoustic energy determined at a plurality of locations over the thorax for two or more of the sub-intervals. An image of for each of these sub intervals may then be determined and displayed sequentially on a display monitor. This generates a movie showing dynamic changes occurring in the acoustic energy in the respiratory tract over the time interval.

### SUMMARY OF THE INVENTION

In the following description and set of claims, two explicitly described, calculable, or measurable variables are considered equivalent to each other when the two variables are proportional to one another.

In its first aspect, the present invention provides a method for managing an interventional pulmonology procedure. As used herein, the term “interventional pulmonology procedure” refers to any interventional procedure that affects the flow of air in the respiratory tract during respiration. Such procedures include, for example, balloon dilation, laser bronchoscopy, cryotherapy, electrocautery, brachytherapy, photodynamic therapy, and stent placement and administering medication into target areas in the lungs.

In accordance with the invention, microphones are affixed to the body surface of an individual at a plurality of locations over the thorax. At one or more times prior to carrying out an interventional pulmonary procedure, signals indicative of respiratory tract sounds are recorded. The signals are analyzed in order to generate one or more images of the respiratory tract indicative of the airflow in the individual's respiratory tract before the procedure is carried out. At one or more times following the interventional procedure, signals indicative of respiratory tract sounds are recorded again. The signals are analyzed in order to generate one or more images of the respiratory tract indicative of the airflow in the individual's respiratory tract after the procedure has been carried out. Images obtained before and after the procedure are compared in order to determine whether a change in the airflow has occurred as a result of the intervention.

The system of the invention includes a plurality of N transducers (microphones) configured to be attached to an essentially planar region R of an individual's back or chest over the thorax. The transducers are typically embedded in a matrix that permits them to be affixed easily on the individual's skin. Such a matrix may typically be in the form of a vest or garment for facilitating affixing of the microphones on the skin over the thorax. As may be appreciated, different matrices may be used to accommodate individuals of different sizes, different ages, sexes, etc.

The system of the invention further comprises a display device for simultaneously displaying at least one image obtained before the interventional procedure and at least one image obtained after the procedure in order to allow a user to compare the images and determine whether a change in the respiratory tract airflow occurred following the procedure.

In a preferred embodiment, a first time interval before and procedure is carried out and a recent time interval after the procedure is carried out are each divided into a plurality of subintervals, and an image is produced for each subinterval. This generates a first sequence of images indicative of respiratory tract airflow prior to the procedure, and a second sequence of images indicative of airflow after the procedure. When the images in a sequence are displayed sequentially on the display device, the sequence is displayed as a movie of respiratory tract airflow over the time interval. Thus, in a most preferred embodiment, a movie of respiratory tract airflow is obtained before and after the interventional procedure, and the two movies are displayed simultaneously on the display device.

Positions in the region R are indicated by two-dimensional position vectors x=(x¹,x²) in a two-dimensional coordinate system defined in the planar region R. The ith transducer, for i=1 to N, is fixed at a position xᵢ in the region R and generates a signal, denoted herein by P(xᵢ,t), indicative of pressure waves in the body arriving at xᵢ.

Any known method for generating images of the respiratory tract from the P(xᵢ,t), may be used in accordance with the invention. In a preferred embodiment, images of the respiratory tract are obtained as disclosed in U.S. Pat. No. 6,887,208 to Kushnir et al. This patent discloses a system and method for calculating an average acoustic energy in the region at a plurality of locations x in the region R over a time interval from t₁ to t₂, denoted herein by \tilde{P}(x,t₁,t₂), from the signals P(xᵢ,t) and generating an image of the lungs from the \tilde{P}(x,t₁,t₂).

In one embodiment of the invention, an average acoustic energy over a time interval from t₁ to t₂ is obtained at a position of one of the microphones using the algebraic expression

$$\tilde{P} ( {x}_{i} , {t}_{1} , {t}_{2} ) = {\int }_{t 1}^{t 2} {P}^{2} ( {x}_{i} , t ) ⅆ t \qquad ( 1 )$$

where xᵢ is the position of the microphone.

In a more preferred embodiment, an average acoustic energy \tilde{P}(xᵢ,t₁,t₂) over a time interval from t₁ to t₂ is obtained at a plurality of positions xᵢ of the microphones, for example using Equation (1), and then calculating \tilde{P}(x,t₁,t₂) at other locations x by interpolation of the \tilde{P}(xᵢ,t₁,t₂) using any known interpolation method.

In a most preferred embodiment, the interpolation is performed to obtain an average acoustic energy \tilde{P}(x,t₁,t₂) at a position x=(x¹,x²) in the surface R using the algebraic expression:

$$\tilde{P} ( x , {t}_{1} , {t}_{2} ) = {\sum }_{i = 1}^{N} \tilde{P} ( {x}_{i} , {t}_{1} , {t}_{2} ) g ( x , {x}_{i} , σ ) \qquad ( 2 )$$

where g(x,xᵢ,σ) is a kernel satisfying

$$\begin{matrix}{∇}^{2} g = \frac{\partial g}{\partial σ} & ( 3 ) \\ {\sum }_{i = 1}^{N} g ( x , {x}_{i} , σ ) is approximately equal to 1 & ( 4 )\end{matrix}$$

and where xᵢ=(xᵢ¹,xᵢ²) is the position of the ith microphone and σ is a selectable parameter.

For example, the kernel

$$g ( x , {x}_{i} , σ ) = Exp - ( \frac{{( {x}^{1} - {x}_{i}^{1} \sqrt{σ} )}^{2}}{2 σ} ) \cdot Exp - ( \frac{{( {x}^{2} - {x}_{i}^{2} \sqrt{σ} )}^{2}}{2 σ} ) \qquad ( 5 )$$

may be used.

It will also be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

Thus, in its first aspect, the present invention provides a system for assessing an interventional pulmonology procedure comprising:
(a) a plurality of N transducers, each transducer configured to be fixed on a surface of the individual over an individual's respiratory tract, the ith transducer being fixed at a location xᵢ and generating a signal P(xᵢ, t) indicative of pressure waves at the location xᵢ; for i=1 to N;
(b) and a processor configured to
   (i) receive the signals P(xᵢ,t)
   (ii) generate a first sequence of one or more images indicative of airflow in at least a portion of the respiratory tract from the signals P(xᵢ,t) over a first time interval from a first time t₁ to a second time t₂, wherein the time interval from t₁ to t₂ occurs before the interventional pulmonology procedure is carried out;
   (iii) generate a second sequence of one or more images indicative of airflow in at least a portion of the respiratory tract from the signals P(xᵢ,t) over a second time interval from a third time t₃ to a fourth time t₄, wherein the time interval from t₃ to t₄ occurs after the interventional procedure has been carried out;
   (iv) display the first and second sequences of images simultaneously on a display device;
(c) the display device simultaneously displaying the sequences of images of the respiratory tract generated by the processor.

In its second aspect, the invention provides a method for assessing an interventional pulmonology procedure in an individual, comprising:
(a) Obtaining a first sequence of one or more images indicative of airflow in at least a portion of the individual's respiratory tract prior to carrying out the interventional pulmonology procedure,
(b) Obtaining a second sequence of one or more images indicative of airflow in at least a portion of the individual's respiratory tract after carrying out the interventional procedure; and
(c) Comparing the first and second sequence of images to determine a change in airflow in the respiratory tract following the interventional pulmonary procedure;
wherein one or more images are obtained in a process comprising:
   (i) affixing a plurality of N transducers, on a surface of the individual over the individual's respiratory tract, the ith transducer being fixed at a location x;
   (ii) obtaining a signal P(xᵢ,t)indicative of pressure waves at the location xᵢ; for i=1 to N;
   (iii) generating the image from the obtained signals P(xᵢ,t).

In its third aspect, the invention provides a computer program comprising computer program code means for performing all the steps of the method of the invention when said program is run on a computer.

In its fourth aspect, the invention provides a computer program comprising computer program code means for performing all the steps of the method of the invention when said program is run on a computer embodied on a computer readable medium.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for managing interventional pulmonology in accordance with one embodiment of the invention;

FIG. 2 shows a flow chart for carrying out a method of managing interventional pulmonology in accordance with one embodiment of the invention;

FIG. 3a shows an image of the respiratory tract of an individual in which a tumor obstructs the right main bronchus restricting the flow of air into the right lung and FIG. 3b shows an image respiratory tract of the same individual following deployment of a stent in the right main bronchus;

FIG. 4a shows an image of the respiratory tract of an individual in which a tumor obstructs the right main bronchus restricting the flow of air into the right lung and FIG. 4b shows an image respiratory tract of the same individual following laser resection of the right main bronchus; and

FIG. 5a shows an image of the respiratory tract of an individual in which a foreign body obstructs the left main bronchus restricting the flow of air into the left lung and FIG. 5b shows an image respiratory tract of the same individual following removal of the foreign body.

### DETAILED DESCRIPTION OF A PREFERRED EMBODIMENT OF THE INVENTION

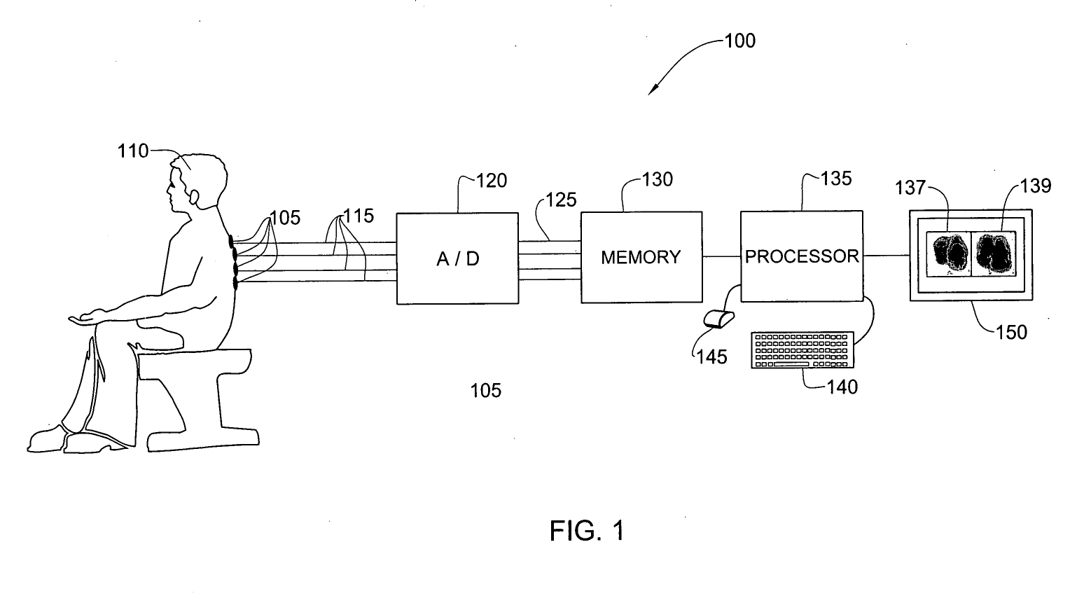

FIG. 1 shows a system generally indicated by 100 for carrying out and assessing a pulmonary interventional procedure in accordance with one embodiment of the invention. A plurality of N sound transducers 105, of which four are shown, are applied to a planar region of the chest or back skin of individual 110. The transducers 105 may be applied to the subject by any means known in the art, for example using an adhesive, suction, or fastening straps. Each transducer 105 produces an analog voltage signal 115 indicative of pressure waves arriving to the transducer. The analog signals 115 are digitized by a multichannel analog to digital converter 120. The digital data signals P(xᵢ,t) 125, represent the pressure wave at the location xᵢ of the ith transducer (i=1 to N) at time t. The data signals 125 are input to a memory 130. Data input to the memory 130 are accessed by a processor 135 configured to process the data signals 125. The signals 125 may be denoised by filtering components such having frequencies outside of the range of lung sounds, for example, vibrations due to movement of the individual. Each signal 125 may also be subject to band pass filtering so that only components in the signal within a range of interest are analyzed.

An input device such as a computer keyboard 140 or mouse 145 is used to input relevant information relating to the examination such as personal details of the individual 110. The input device 140 may also be used to input values of a first pair of times t₁ and t₂ where the time interval from t₁ to t₂ occurs before the interventional procedure has been carried out. The processor 135 performs an analysis of the signals P(xᵢ,t) from t₁ to t₂ so as to generate one or more images 137 indicative of airflow in the individual's respiratory tract prior to the interventional procedure. In a preferred embodiment, the analysis involves determining an average acoustic energy \tilde{P}(x,t₁,t₂)over the time interval from t₁ to t₂ at least one location x in the region R in a calculation involving at least one of the signals P(xᵢ,t). The input device 140 may also be used to input values at a second pair of times t₃ and t₄, where the time interval t₃ to t₄ occurs after the interventional procedure has been carried out. The processor 135 then performs an analysis of the signals P(xᵢ,t) from t₃ to t₄ to generate one or more images 139 indicative of airflow in the individual's respiratory tract after the interventional procedure has been carried out. The images 137 and 139 of the respiratory tract generated before and after the interventional procedure are displayed simultaneously on a display device 150, such as a monitor screen.

In a more preferred embodiment, the time intervals from t₁ to t₂ and from t₃ to t₄ are divided into a plurality of subintervals, and an image is generated from the signals P(xᵢ,t) for each subinterval. This generates movies of respiratory tract airflow before and after the interventional procedure that are displayed simultaneously on the display device 150.

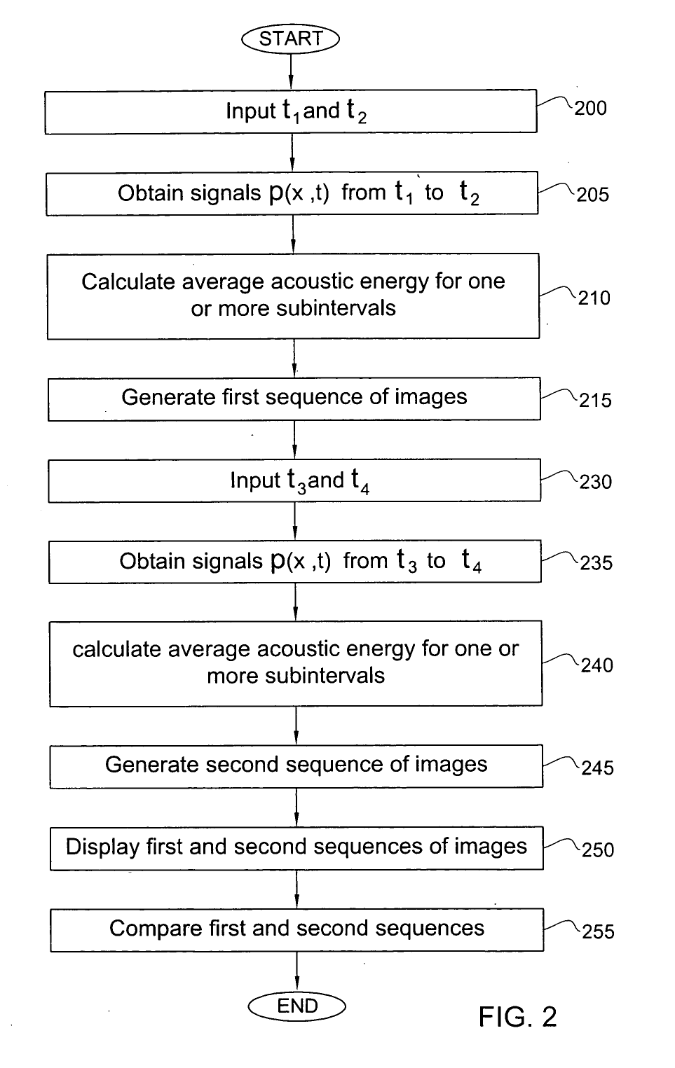

FIG. 2 shows a flow chart for managing a pulmonary interventional procedure in accordance with one embodiment of the method of the invention. In step 200 values of times t₁ and t₂ are input to the processor 135 using the input devices 140 or 145, where the time interval from t₁ and t₂ occurs before the pulmonary interventional procedure is carried out. In step 205 the signals P(xᵢ,t) are obtained from N transducers placed at predetermined locations xᵢ for i from 1 to N in a region R overlying the lungs over the time interval from a time t₁ to time t₂. In step 210, an average acoustic energy is calculated while the individual is breathing at a plurality of locations x in the region R for one or move subintervals of the time interval t₁ to t₂. In step 215 a first sequence of one or more images is generated of the lungs from the average acoustic energies indicative of airflow in the individual's respiratory tract prior to carrying out the interventional procedure. In step 230 values of times t₃ and t₄ are input to the processor 135 using the input devices 140 or 145, where the time interval from t₃ to t₄ occurs after the pulmonary interventional procedure has been carried out. In step 235, signals P(xᵢ,t) are obtained from the N transducers placed at the locations xᵢ for i from 1 to N in the region R. In step 240, an average acoustic energy for one or more subintervals is calculated at a plurality of locations x in the region R for one or more subintervals of time interval t₃ to t₄. In step 245 a second sequence of images is generated of the lungs from the average acoustic energies indicative of airflow in the individual's respiratory tract following the interventional procedure. In step 250 the first and second images are displayed simultaneously on the display device 150. In step 255, the first and second images are compared and the effect of the interventional procedure is assessed.

### EXAMPLES

The system and method of the invention were used to perform a pulmonary interventional procedure.

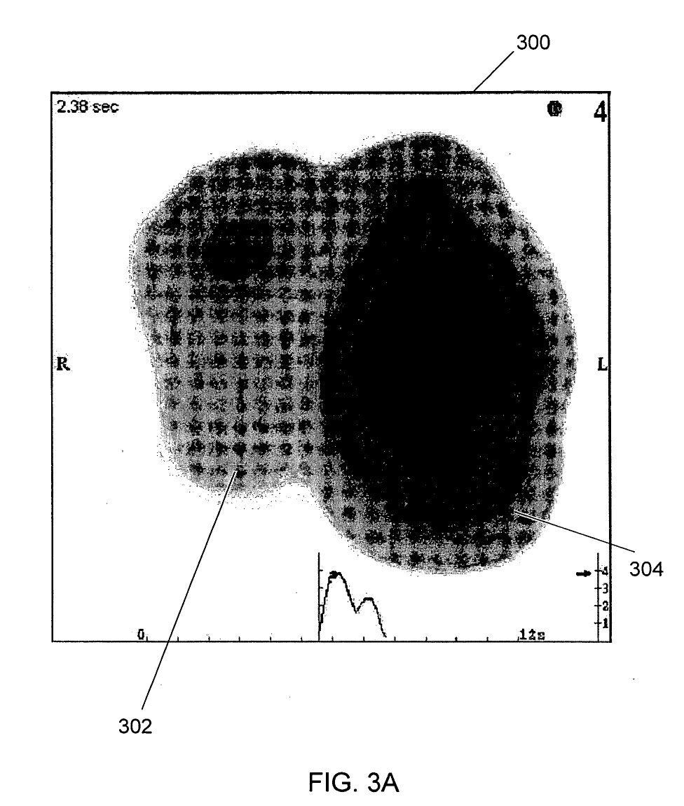

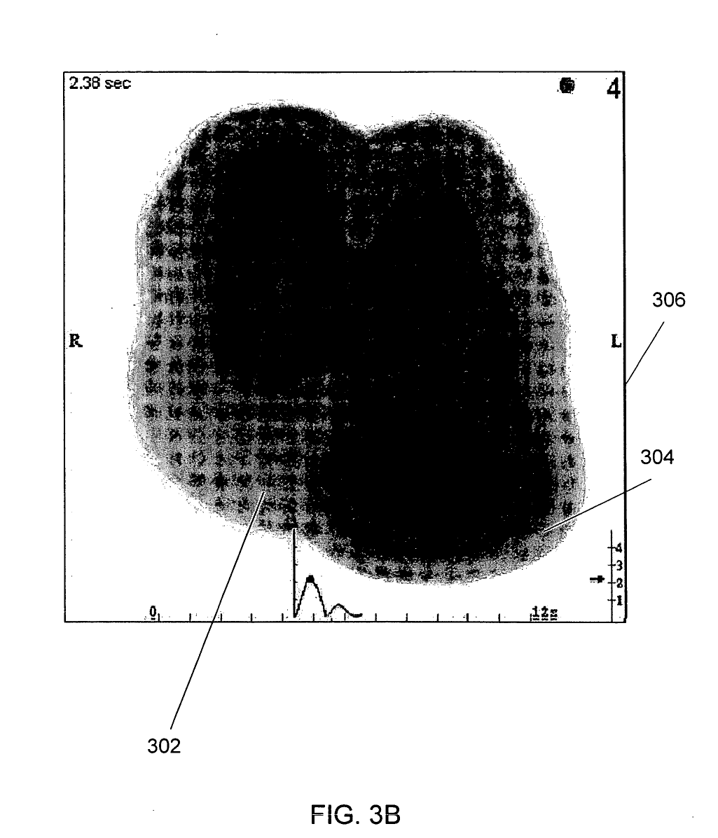

FIG. 3a shows an image 300 of the right lung 302 and the left lung 304 of an individual indicative of airflow in the lungs obtained by the method disclosed in U.S. Pat. No. 6,887,208 to Kushnir et al using equations (2) , (3) and (4) above with σ=36. The image 300 was obtained in mid-inspirum. A tumor obstructs the right main bronchus restricting the flow of air into the right lung 302. Airflow in the left lung 304 appears to be unobstructed. FIG. 3b shows an image 306 of the lungs 302 and 304 of the same individual in mid-inspirum obtained by the same method following deployment of a stent in the right main bronchus. Comparison of the image 300 FIG. 3a with the image 306 of FIG. 3b shows that following stent deployment, flow of air into the right lung 302 was significantly increased.

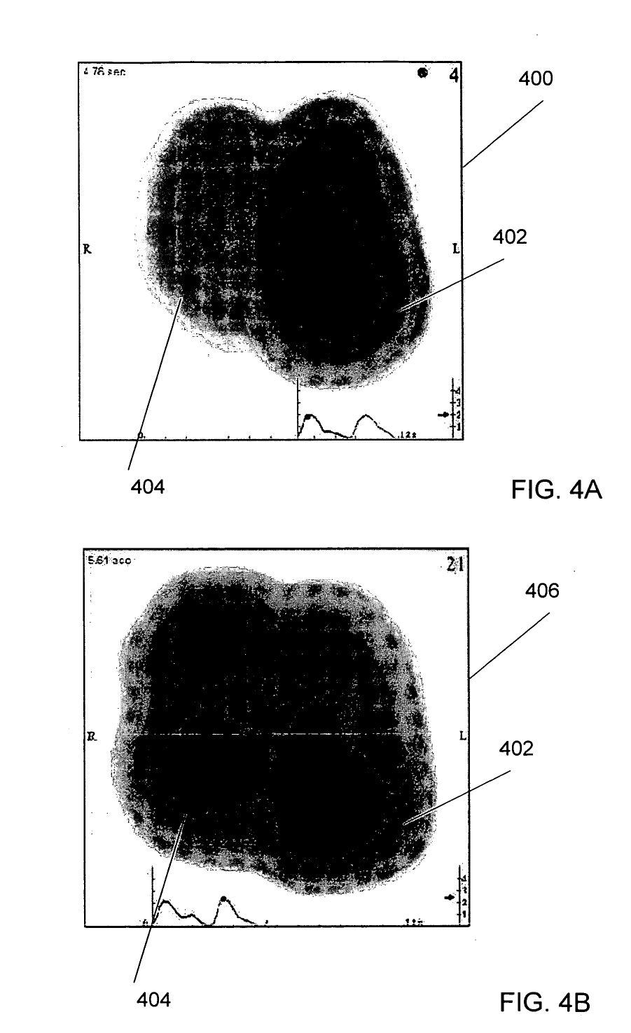

FIG. 4a shows an image 400 of the left lung 402 and the right lung 404 of an individual indicative of airflow in the lungs obtained by the method disclosed in U.S. Pat. No. 6,887,208 to Kushnir et al using equations (2) , (4) and (4) above with σ=36. The image 400 was obtained in mid-inspirum. A tumor obstructs the right main bronchus restricting the flow of air into the right lung 402. Airflow in the left lung 404 appears to be unobstructed. FIG. 4b shows an image 406 of the lungs 402 and 404 of the same individual in mid-inspirum obtained by the same method following laser resection of the right main bronchus. Comparison of the image 400 FIG. 4a with the image 406 of FIG. 4b shows that following the laser resection, flow of air into the right lung 402 was significantly increased.

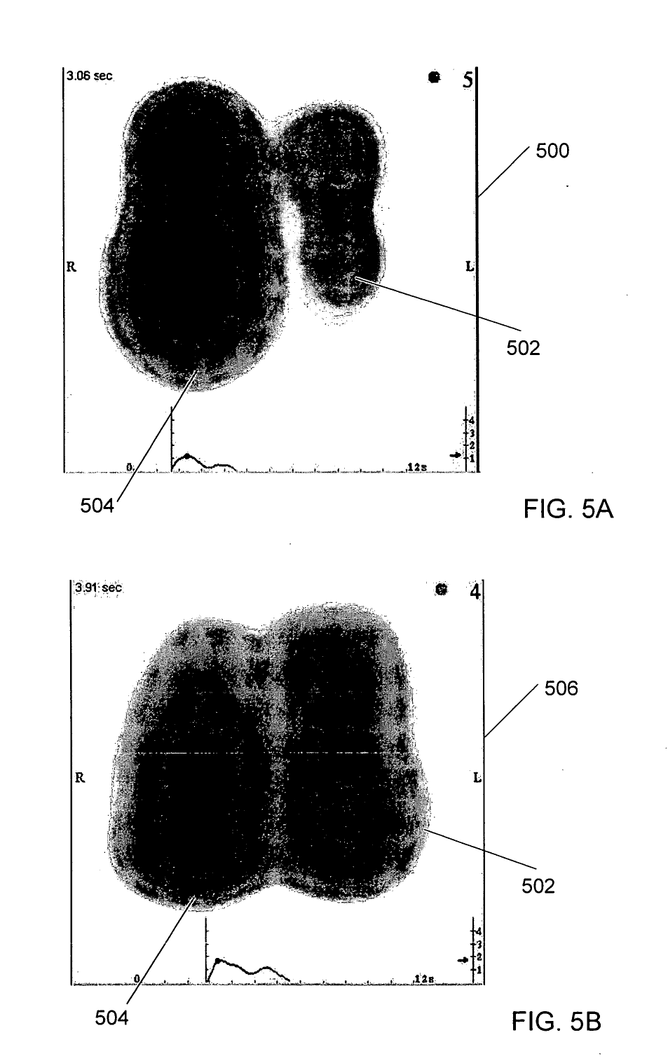

FIG. 5a shows an image 500 of the left lung 502 and the right lung 504 of an individual indicative of airflow in the lungs obtained by the method disclosed in U.S. Pat. No. 6,887,208 to Kushnir et al using equations (2) , (5) and (4) above with σ=36. The image 500 was obtained in mid-inspirum. A foreign body obstructs the left main bronchus restricting the flow of air into the left lung 502. Airflow in the right lung 504 appears to be unobstructed. FIG. 5b shows an image 506 of the lungs 502 and 504 of the same individual in mid-inspirum obtained by the same method following removal of the foreign body from the left main bronchus. Comparison of the image 500 FIG. 5a with the image 506 of FIG. 5b shows that following removal of the foreign body, flow of air into the left lung 502 was significantly increased.

1. A system for assessing an interventional pulmonology procedure comprising:
(a) a plurality of N transducers, each transducer configured to be fixed on a surface of the individual over an individual's respiratory tract, the ith transducer being fixed at a location xᵢ and generating a signal P(xᵢ,t) indicative of pressure waves at the location xᵢ; for i=1 to N;
(b) and a processor configured to
(i) receive the signals P(xᵢ,t)
(ii) generate a first sequence of one or more images indicative of airflow in at least a portion of the respiratory tract from the signals P(xᵢ,t) over a first time interval from a first time t₁ to a second time t₂, wherein the time interval from t₁ to t₂ occurs before the interventional pulmonology procedure is carried out;
(iii) generate a second sequence of one or more images indicative of airflow in at least a portion of the respiratory tract from the signals P(xᵢ,t) over a second time interval from a third time t₃ to a fourth time t₄, wherein the time interval from t₃ to t₄ occurs after the interventional procedure has been carried out;
(iv) display the first and second sequences of images simultaneously on a display device;
(c) the display device simultaneously displaying the sequences of images of the respiratory tract generated by the processor.

2. The system according to claim 1 wherein the processor is configured to generate one or more images in an algorithm involving calculation of an average acoustic energy \tilde{P}(x,t₁,t₂) at a plurality of positions x over one or more subintervals of the \tilde{P} being calculated in an algorithm involving at least one of the signals.

3. The system according to claim 2 wherein the average acoustic energy \tilde{P} over a time subinterval from tₖ₁ to tₖ₂ is determined at a location xᵢ of a transducer using the algebraic expression:
$$\tilde{P} ( {x}_{i , {t}_{k 1} {t}_{k 2}} ) = {\int }_{{tk}_{1}}^{{tk}_{2}} {P}^{2} ( {x}_{i} , t ) ⅆ t .$$

4. The system according to claim 3 wherein the function \tilde{P} is determined at one or more locations x in an algorithm comprising:
(a) determining an average acoustic energy \tilde{P}(xᵢ,tₖ_{₁}tₖ_{₂}) over a time subinterval from tₖ_{₁} to tₖ_{₂} at a plurality of locations xᵢ of transducers; and
(b) determining an average acoustic energy \tilde{P}(xᵢ,tₖ_{₁}tₖ_{₂}) at at least one location x by interpolation of the determined \tilde{P}(xᵢ,t₁,t₂).

5. The system according to claim 4 wherein an average acoustic energy \tilde{P}(xᵢ,tₖ_{₁}tₖ_{₂}) is determined over a time interval from tₖ_{₁} to tₖ_{₂} at a plurality of locations xᵢ of transducers using the algebraic expression:
$$\tilde{P} ( {x}_{i , {t}_{k 1} {t}_{k 2}} ) = {\int }_{{tk}_{1}}^{{tk}_{2}} {P}^{2} ( {x}_{i} , t ) ⅆ t .$$

6. The system according to claim 5 wherein an average acoustic energy is determined at least one location x by interpolation of the determined \tilde{P}(xᵢ,tₖ_{₁}tₖ_{₂}) using the algebraic expression:
$$\tilde{P} ( x , {t}_{{k}_{1}} {t}_{{k}_{2}} ) = {\sum }_{i = 1}^{N} \tilde{P} ( x , {t}_{{k}_{1}} {t}_{{k}_{2}} ) g ( x , {x}_{i} s ) \qquad ( 2 )$$
where g(x,xᵢ,σ) is a kernel satisfying
$$\begin{matrix}{∇}^{2} g = \frac{\partial g}{\partial σ} & ( 3 ) \\ {\sum }_{i = 1}^{N} g ( x , {x}_{i} , σ ) is approximately equal to 1. & ( 4 )\end{matrix}$$

7. The system according to claim 6 wherein at least one of the first sequence of images and the second sequence of images is a movie indicative of airflow in the at least portion of the respiratory tract.

8. A method for assessing an interventional pulmonology procedure in an individual, comprising:
(a) Obtaining a first sequence of one or more images indicative of airflow in at least a portion of the individual's respiratory tract prior to carrying out the interventional pulmonology procedure,
(b) Obtaining a second sequence of one or more images indicative of airflow in at least a portion of the individual's respiratory tract after carrying out the interventional procedure; and
(c) Comparing the first and second sequence of images to determine a change in airflow in the respiratory tract following the interventional pulmonary procedure;
wherein one or more images are obtained in a process comprising:
(i) affixing a plurality of N transducers, on a surface of the individual over the individual's respiratory tract, the ith transducer being fixed at a location x;
(ii) obtaining a signal P(xᵢ,t) indicative of pressure waves at the location xᵢ; for i=1 to N;
(iii) generating the image from the obtained signals P(xᵢ,t).

9. The method according to claim 8 further comprising calculating an average acoustic energy \tilde{P}(x,t₁,t₂) at a plurality of positions x over a time interval from a first time t₁ to a second time t₂, \tilde{P} being determined in an algorithm involving at least one of the signals P(xᵢ,t), and generating an image of the respiratory tract based upon the \tilde{P}(x,t₁,t₂).

10. The method according to claim 9 wherein the average acoustic energy \tilde{P} over a time interval from t₁ to t₂ is determined at a location xᵢ of a transducer using the algebraic expression:
$$\tilde{P} ( {x}_{i} , {t}_{1} , {t}_{2} ) = {\int }_{t 1}^{t 2} {P}^{2} ( {x}_{i} , t ) ⅆ t .$$

11. The method according to claim 9 wherein the function \tilde{P} is determined at one or more locations x in an algorithm comprising:
(c) determining an average acoustic energy \tilde{P}(xᵢ,t₁,t₂) over a time interval from t₁ to t₂ at a plurality of locations xᵢ of transducers; and
(d) determining an average acoustic energy \tilde{P}(x,t₁,t₂) at at least one location x by interpolation of the determined \tilde{P}(xᵢ,t₁,t₂).

12. The method according to claim 11 wherein an average acoustic energy \tilde{P}(xᵢ,t₁,t₂) is determined over a time interval from t₁ to t₂ at a plurality of locations xᵢ of transducers using the algebraic expression:
$$\tilde{P} ( {x}_{i} , {t}_{1} , {t}_{2} ) = {\int }_{t 2}^{t 2} {P}^{2} ( {x}_{i} , t ) ⅆ t .$$

13. The method according to claim 12 wherein an average acoustic energy is determined at at least one location x by interpolation of the determined \tilde{P}(xᵢ,t₁,t₂) using the algebraic expression:
$$\tilde{P} ( x , {t}_{1} , {t}_{2} ) = {\sum }_{i = 1}^{N} \tilde{P} ( {x}_{i} , {t}_{1} , {t}_{2} ) g ( x , {x}_{i} , σ ) \qquad ( 2 )$$
where g(x,xᵢ,σ) is a kernel satisfying
$$\begin{matrix}{∇}^{2} g = \frac{\partial g}{\partial σ} & ( 3 ) \\ {\sum }_{i = 1}^{N} g ( x , {x}_{i} , σ ) is approximately equal to 1. & ( 4 )\end{matrix}$$

14. The method according to claim 14 wherein g(x,νᵢσ) is the kernel
$$g ( x , {x}_{i} , σ ) = Exp - ( \frac{{( {x}^{1} - {x}_{i}^{1} \sqrt{σ} )}^{2}}{2 σ} ) \cdot Exp - ( \frac{{( {x}^{2} - {x}_{i}^{2} \sqrt{σ} )}^{2}}{2 σ} ) . \qquad ( 5 )$$

15. The method according to claim 14, wherein at least one of the first sequence of images and the second sequence of images is a movie indicative of airflow in the at least portion of the respiratory tract.

16. The method according to claim 15, further comprising simultaneously displaying the first and second sequences of images on a display device.

17. A computer program comprising computer program code means for performing all the steps of claim 16 when said program is run on a computer.

18. A computer program as claimed in claim 17 embodied on a computer readable medium.
